(19) United States
(12) Patent Application Publication
(10) Pub. No.: US 2013/0266721 A1
(43) Pub. Date: Oct. 10, 2013

(54) Preparation of controlled drug release porous hydroxyapatite microspheres with interconnected pore channels

(71) Applicant: Tapash Ranjan Rautray, Bhubaneswar (IN)

(72) Inventor: Tapash Ranjan Rautray, Bhubaneswar (IN)

(21) Appl. No.: 13/441,000

(22) Filed: Apr. 6, 2012

(51) Int. Cl. A61K 9/16

(52) U.S. Cl. CPC A61K 9/1682
USPC 427/2.21

(57) ABSTRACT

A process for the preparation of controlled drug release porous hydroxyapatite microspheres, in the form of granules, consisting of interconnected pore channels with high porosity. It comprises the steps of mixing nano-hydroxyapatite powder with camphene to form a homogenous mixture followed by mixing said homogenous mixture with gelatin solution to form a complex mixture. This complex mixture is dispersed in cold stirring oil in a beaker until said dispersed granules hardens to form green bodies. These green bodies are subjected to heat treatment to obtain porous hydroxyapatite microspheres, subjecting the said porous hydroxyapatite microspheres to antibiotics encapsulation.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

i. Field of the Invention

The present invention relates generally to preparation of porous hydroxyapatite microspheres and, more particularly, relates to preparation of drug encapsulated controlled release porous hydroxyapatite microspheres with interconnected pore channels.

ii. Description of the Related Art

The application of autograft for repairing bone defect is limited by the shortage of available tissues and the inconvenience caused by resulting donor site morbidity. Allograft and xenograft, on the other hand, often elicit undesired immunological rejection from the host. Because of the numerous complications associated with the tissue-based grafting materials, synthetic bone substitutes have been developed as alternatives of the materials for repairing bone defect.

Hydroxyapatite received considerable attention as bone graft substitute material because of its excellent biocompatibility, bioactivity, and osteoconductivity.

Although hydroxyapatite powder remains the best form of choice for filling small irregular defects, the therapeutic effect of the filling implant is lost by migration of particles from the defect site. Furthermore, it was too difficult to be handled and kept in place compactly for convenient fabrication and hence the hydroxyapatite powder was given spherical granular form to overcome these problems by adding suitable binder.

Existing processes for making hydroxyapatite microspheres using freeze-casting method using camphene as a porogen show irregular or spherical geometry because the granules are made of breaking the pre-fabricated porous blocks. Such an irregular geometry seems to be less than ideal because it can induce inflammatory reactions in body soft tissue.

The shapes of hydroxyapatite microspheres used as bone graft substitutes have been reported to play a crucial role in bone formation. Moreover, porosity, pore size, and interconnectivity have been reported to play vital role in bone ingrowth. Hydroxyapatite microspheres are thus one of the most important biomaterials in optimizing bone reconstruction ability. Spherical hydroxyapatite granules have closer packing and greater flexibility in filling different geometric cavity because they have large specific surface area with good flowability.

Hydroxyapatite microspheres can have varying porosities on them for optimizing ingrowth of different tissues and cells. It is proposed that 30 μm sized pore may be the smallest pore that can be used for osteoconduction, and the 150 μm sized pore as the optimal pore.

Pore forming on calcium phosphate materials are carried out by using (i) replica technique, (ii) sacrificial template technique, and (iii) direct foaming technique. Camphene based pore formation comes under the second category.

Spherical hydroxyapatite granules are filled in the bone defect site which form a matrix with uniform pores among hydroxyapatite granules and promotes efficient conduction of bone from granule to granule. Packed hydroxyapatite granules have bimodal pore size distribution structure of intergranular and intragranular pores.

Owing to their physicochemical and biological properties, hydroxyapatite microspheres are considered as a potential material for bone drug delivery system; using a bioactive matrix, can release a therapeutic agent in situ to produce an action associating the osteoconductivity of the material.

### SUMMARY OF THE INVENTION

In accordance with one aspect of the present invention, methods are provided for the preparation of porous hydroxyapatite microspheres with interconnected pore channels by emulsion method.

In another aspect, the porous hydroxyapatite microspheres are encapsulated with controlled release drugs like gentamicin.

The process starts with the steps of mixing hydroxyapatite-camphene mixture with gelatin solution, dispersing the said solution in stirring oil kept at low temperature, and allowing the dispersed granules to harden. Spherical granules are formed there and subsequently they are heat treated to obtain porous hydroxyapatite microspheres with interconnected pore channels. Drugs such as gentamicin encapsulation into hydroxyapatite microspheres are carried out in phosphate buffer saline at room temperature for 24 hrs.

These porous hydroxyapatite microspheres can be used as bone graft substitutes with controlled drug release capabilities.

### BRIEF DESCRIPTION OF THE SEVERAL VIEWS OF THE DRAWING

FIG. 1. Scanning electron microscopy image of porous hydroxyapatite microsphere (Original magnification ×50).

FIG. 2. Micro CT image of hydroxyapatite microsphere.

FIG. 3. Scanning electron microscopy image of MG63 cells cultured on porous hydroxyapatite microspheres (Original magnification ×1000).

FIG. 4. In vitro release of antibiotics from the porous hydroxyapatite microspheres.

### DETAILED DESCRIPTION OF THE INVENTION

This invention relates to preparation of hydroxyapatite microspheres with drug delivery capabilities. More particularly, this invention relates to the preparation of controlled drug release porous hydroxyapatite microspheres with interconnected pore channels.

Hydroxyapatite microspheres with drug delivery capabilities having diameter of 1000-2000 μm diameter with interconnected pore channels with high porosity may be used as bone graft substitutes in orthopaedics. These porous hydroxyapatite microspheres may carry antibiotic drugs such as gentamicin.

Camphene is non-toxic and easily sublimates under ambient conditions. Camphene with a melting temperature of 48-52° C. and a density of 0.85 g/cc was selected as a porogen. Camphene forms dendrites when solidified under an appropriate temperature gradient. Moreover, the solidified camphene easily sublimates under atmospheric conditions within 6 hrs. Therefore, camphene was selected as a porogen to generate interconnected pores in hydroxyapatite granules.

Gelatin has been reported to have good biocompatibility which is sensitive to temperature and concentration such that lower temperatures and higher concentrations lead to faster gel formation and stronger gels. If the concentration of gelatin is too high, spherical form is difficult to produce due to reduction of flow. If that is too low, in contrast, it also has some problems with hardening of granules and low bond strength among hydroxyapatite particles. In view of these facts, 10% gelatin solution was used for the preparation of hydroxyapatite microspheres.

The microstructure, pore size and interconnectivity of hydroxyapatite microspheres directly affect the porosity features. Porosity of hydroxyapatite microspheres greatly contribute to both its mechanical and biological properties and their increased surface area provides a greater means of environmental interaction. It is known that hydroxyapatite microspheres with higher porosity and lower density provide greater surface area for vascularization and bone in-growth as compared with the dense microspheres because of their resorbability and osteoconductivity. Human osteoblasts can penetrate into pores greater than 20 μm in size and at porosity greater than 30%. However, it is the degree of interconnectivity of porous hydroxyapatite granules that is more significant than the sizes of pores or the porosity in that the vascular network needed for new bone penetration and formation is chiefly affected by the hydroxyapatite granular interconnectivity. Therefore, the porosity must be interconnected to allow the ingrowth of cells, vascularization, and diffusion of nutrients. In highly porous and interconnected scaffolds, fibrovascular tissue in-growth starts by day 3 or 4 and by 28 days this ingrowth completes throughout the scaffold and apposition of bone against the pore wall begins.

It is therefore an aspect of the present invention to provide a process for the preparation of hydroxyapatite microspheres that are spherical, highly porous with interconnected pore channels with controlled drug release capabilities.

i. Preparation Procedure

Porous hydroxyapatite microspheres are formed by mixing nano-sized hydroxyapatite powder with various amounts of camphene (Sigma-Aldrich) porogen with camphene to hydroxyapatite ratio of 90% w/w. 10% gelatin (Sigma-Aldrich, St. Louis, Mo., USA) solution was added to the camphene+hydroxyapatite mixture which was prepared by adding 10% gelatin by volume of the solution containing type B bovine skin gelatin (Sigma-Aldrich, St. Louis, Mo., USA) in 100 ml distilled water containing 2% polyvinyl alcohol (Daejung Chemicals & Metals Co. Ltd, Shiheung, Korea) at 50° C., 0.2% Triton X-100 (Amresco Inc., Solon, Ohio, USA) and 0.3% ploy ammonium salt (Duramax D-3005, Rohm and Haas, Philadelphia, Pa., USA). This hydroxyapatite-camphene-gelatin mixture was dispersed in a beaker containing cold vegetable oil stirred at 150-300 rpm, which was surrounded by ice in a bath. Before dispersing this mixture, the beaker containing the vegetable oil was kept in an ice-cooled bath for 15 mins. for the oil to cool down and after dispersing the mixture, the granules were kept on stirring for about 15 mins. until the granules got hardened. The spherical granules were then removed from the oil, rinsed with ethanol, and then stored at −10 C for 10 mins. and subsequently dried at room temperature for 6 hours. The granules with 1000-2000 μm diameters were separated by using standard sieves set. The hydroxyapatite granules were sintered at 1250 C for 3 hrs. Encapsulation of antibiotics such as gentamicin into hydroxyapatite microspheres was carried out in phosphate buffer saline with pH 7.4 at room temperature for 24 hrs. 10 gm of microspheres was immersed in 1 litre of phosphate buffered saline containing 10 gm of gentamicin. After 24 hrs., the microspheres were separated by centrifugation and dried at room temperature for 48 hrs. to obtain the controlled drug release porous hydroxyapatite microspheres. Taking advantage of the porosity of the microspheres, a greater amount of antibiotics could be incorporated into the microspheres.

ii. Characterization Experiments and in Vitro Studies

The drug encapsulated hydroxyapatite granules were characterized by scanning electron microscope (SEM), micro-computed tomography (microCT) scan, spectrophotometer and porosimeter. It was found that the hydroxyapatite microspheres had interconnected pore channels with porosity of about 63%. Also, the presence of macropores (with sizes >50 μm) in the hydroxyapatite microspheres was confirmed by SEM. These microspheres with interconnected pore channels may be used as bone graft substitute materials with drug delivery capabilities in bone tissue engineering applications.

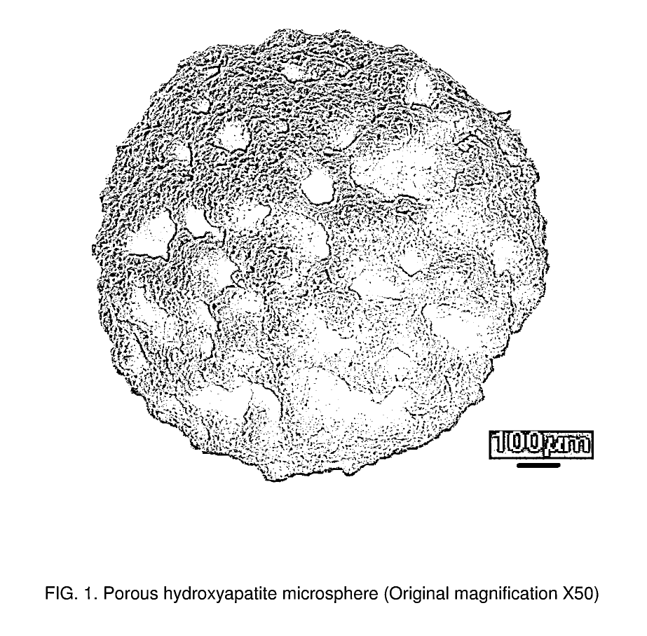

As shown in FIG. 1, large pores (macropores) with sizes of at least 30 μm could be produced in the hydroxyapatite granules. On the other hand, micropores (<10 μm) were not affected with different amount of camphene because they are strongly dependent on the preparation route. The micropores on the surface can greatly enhance the surface roughness and surface area, which improves the cell adhesion, proliferation, phenotype, and the mechanical interlocking with surrounding tissues by biological fixation in vivo. Generally, porosity and roughness are more biologically favorable than smooth surfaces.

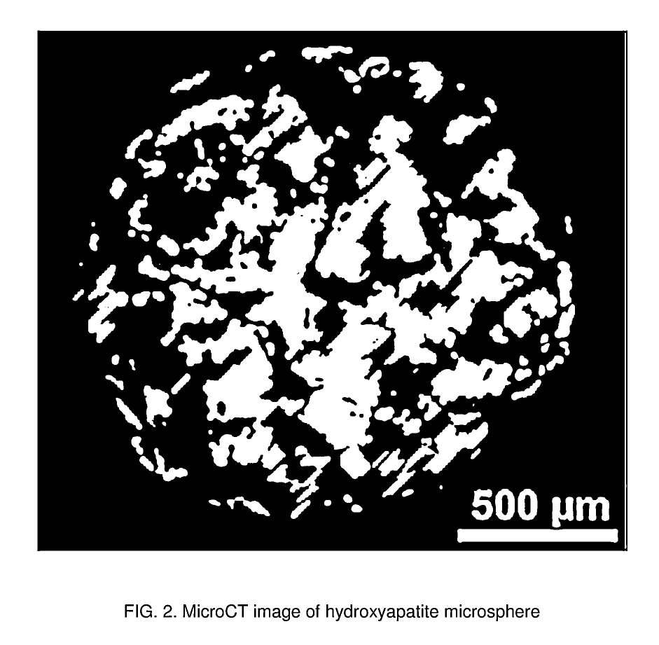

The microCT image as shown in FIG. 2 shows the interconnected pore channels.

MG63 osteoblast-like cell, originally isolated from a human osteosarcoma, was used for the in vitro experiment. The MG63 cell was obtained from the ATCC (American Type Culture Collection) and cultured in DMEM medium (Sigma Aldrich, St Louis, Mo., USA) supplemented with 10% fetal bovine serum (FBS) (PAA Laboratories GmbH, Linz, Austria), 1% penicillin (10,000 units)/streptomycin (10 mg/mL, GibcoBRL, Gaithersburg, Md., USA) and 1% 200 mm l-glutamine (PAA Laboratories GmbH, Linz, Austria) at 37 C in a humidified atmosphere of 5% CO₂ in air. The complete medium was replaced every 2 days and confluent cells were subcultured using the Trypsin-EDTA (0.5 g/L Trypsin and 0.2 g/L EDTA, GibcoBRL, Gaithersburg, Md., USA). After counting, cells were seeded on hydroxyapatite microspheres.

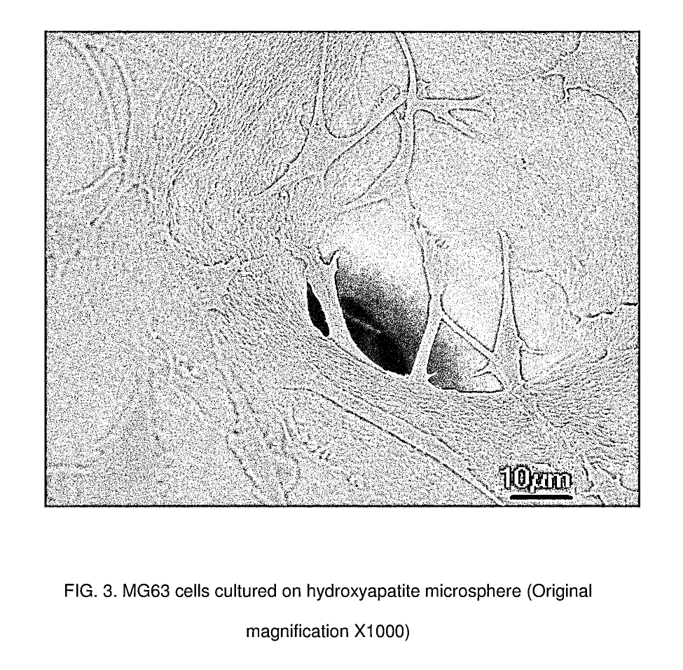

As shown in FIG. 3, the osteoblast cells spread and formed lamellae on the gentamicin encapsulated porous hydroxyapatite microspheres as shown by their extended filopodia and it was observed that the cell attachment and spreading in the pore channels were also very favourable.

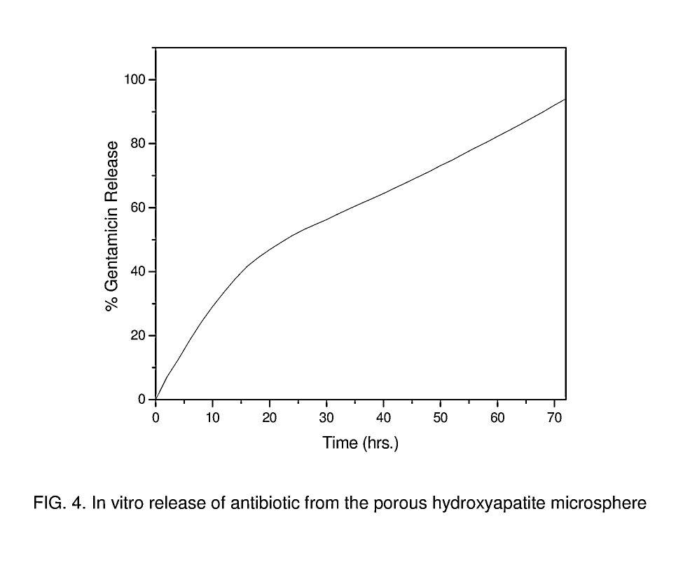

In vitro release of gentamicin from hydroxyapatite microspheres was carried out at 37 C in phosphate buffer saline at pH 7.4. The release medium was collected at an interval of 2 hrs. and replaced with a fresh phosphate buffer saline each time. The collected samples were filtered through a 0.45 mm Millipore filter. The amount of gentamicin released was then measured at 257 nm using a Shimadzu UV-2100S spectrophotometer and is shown in FIG. 4.

Bone to implant contact depends on the direct interactions of bone matrix and osteoblasts with the synthesized biomaterials. Protein adsorption takes place when a biomaterial surface comes in contact with a biological environment. Osteoblast adhesion is therefore essential for bone-biomaterial interactions.

The better cell adhesion and spreading on the hydroxyapatite microspheres was due to the higher roughness and good biocompatibility of porous hydroxyapatite microspheres which accelerated the cell adhesion and spreading.

1. A process for the preparation of controlled drug release porous hydroxyapatite microspheres, in the form of granules, consisting of interconnected pore channels with high porosity, comprising the steps of mixing nano-hydroxyapatite powder with camphene to form a homogenous mixture followed by mixing said homogenous mixture with gelatin solution to form a complex mixture, dispersing said complex mixture in cold stirring oil in a beaker until said dispersed granules hardens to form green bodies, subjecting said green bodies to heat treatment to obtain hydroxyapatite microspheres, subjecting the said hydroxyapatite microspheres to antibiotics encapsulation.

2. The process as claimed in claim 1 wherein camphene was heated at 60 C before mixing with hydroxyapatite powder with a weight ratio of 90% of hydroxyapatite.

3. The process as claimed in claim 1 wherein camphene has a melting temperature of 48-52° C. and a density of 0.85 gm/cc, is used as porogen.

4. The process as claimed in claim 1 wherein gelatin solution is 10% gelatin by volume of the solution containing type B bovine skin gelatin in 100 ml distilled water containing 2% polyvinyl alcohol at 50° C., 0.2% Triton X-100 and 0.3% ploy ammonium salt.

5. The process as claimed in claim 1 wherein 200-500 ml of the vegetable oil stirred at a speed of 150-300 rpm for about 10-30 mins. is used as the emulsifying medium.

6. The process as claimed in claim 1 wherein the oil containing beaker is kept for 10-15 mins. in the ice-cooled bath for rapid solidification before said complex mixture dispersed.

7. The process as claimed in claim 1 wherein green bodies were obtained after separating resultant granules from oil, cleaning with ethanol, storing at −10 C for 10-15 mins. and subsequently drying at room temperature for 6 hours.

8. The process as claimed in claim 1 wherein the green bodies subjected to heat treatment is hydroxyapatite microspheres of 1000-2000 μm diameter separated by using standard sieves.

9. The process as claimed in claim 1 wherein the green bodies subjected to heat treatment is sintered at 1250 C for 3 hrs. at a heating rate of 5 C/min. in an air furnace.

10. The process as claimed in claim 1 wherein said “porous” microspheres refers to the porosity of the hydroxyapatite microspheres not less than 50%.

11. The process as claimed in claim 1 wherein said “spherical” microspheres are the granules having a minimum width which is at least 90% of the maximum width.

12. The process as claimed in claim 5 wherein said emulsifying medium comprises a vegetable oil such as sunflower oil, groundnut oil, or coconut oil, singly or in combination thereof.

13. The process as claimed in claim 13 wherein said antibiotic agent is selected from the class of aminoglycoside or cefalosporin antibiotics.
